SEQUENCE LISTING (9 sequences)

SEQ ID NO: 1 - DNA, 1110 bp, Artificial Sequence
  BlaM-vpr fusion gene insert of pMM310
aagcttggtaccaccatggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggggatccgaacaagccccagaagaccaagggccacagagggagccgcacaatgaatggacactagagcttttagaggagcttaagagagaagctgttagacattttcctaggccatggctacatggcttaggacaacatatctatgaaacttatggagatacttgggcaggagtggaagccataataagaattctgcaacaactgctgtttattcatttcagaattgggtgtcaacatagcagaataggcattattcaacagaggagagcaagaagaaatggagccagtagatcctaactcgag

SEQ ID NO: 2 - protein, 362 aa, Artificial Sequence
  Beta-lactamase-vpr protein
MDPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWGSEQAPEDQGPQREPHNEWTLELLEELKREAVRHFPRPWLHGLGQHIYETYGDTWAGVEAIIRILQQLLFIHFRIGCQHSRIGIIQQRRARRNGASRS

SEQ ID NO: 3 - DNA, 9965 bp, Artificial Sequence
  HIV genomic DNA in pMM326
accctattaccactgccaattacctgtggtttcatttactctaaacctgtgattcctctaaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactagcggccgcaagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatggggtgggagcagtatctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagc

SEQ ID NO: 4 - DNA, 2707 bp, Artificial Sequence
  Glycoprotein gene for R8.1021
gcggccgcaagaaagagcagaagacagtggcaatgagagcgatggggaccaggaagagttggcagcactggagatggggcaccttgctccttgggatgttgatgatctgtagtgctgaagaaaaattgtgggtcacagtctattatggggtacctgtgtggaaagaagcaaccaccactctattttgtgcatcagatgctaaagcatatgacacagaggtacataatgtttgggccacacatgcctgtgtacccacagacccgaatccacaagaagtagtattggaaaatgtgacagaaaattttaacatgtggaaaaatgacatggtagaacagatgcatgaggatataatcagcttgtgggatcaaagtctaaagccatgtgtaaaattaactccactctgtgttactttaaattgcactaatgctaatttaacttactctaatgctactgagaccagtaatagtggaatagcgatagacaaaggagaaataaaaaactgctctttcaatatcaccacaggcataaaaaataagatgcagaaagaatatgctctcttatataaacttgatttaatgccaatagagaataataatgaaagctatacattgataagttgtaacacatcagtcataacacaggcctgtccaaaggtatcctttgaaccaattcccatacatttttgtgccccggctggttttgcgattctaaaatgtaatgataagaagtacaatggaacagggccatgtaacaatgtcagcacagtacaatgtacacatggaattaggccagtagtgtcaactcaattgctgttaaatggcagtctagcagaaaaagaggtaatgattagatctgaaaatttcacggacaatgctaaaaccataatagtacagctgaatgaaactgtaaaaattacttgtataagacccaacaacaatacaagaaaaggtatacatataggaccagggagagcattttatacaacaggaaacataataggagatataagacaagcacattgtaacattagtggagcagattggaataaaactttacatcagatagttaaaaaattaagagaacaattaaggaataatraaacaatagtctttaatcaatcctcagggggggatccagaaattacaatgcacacttttaattgtggaggggaatttttctactgtaacacagcacagttgtttaatagtacttggaatgttactcaagagccaaatatcgctaatggaacaatcacactcccatgcagaataaaacaaattataaacagatggcaagaagtaggaaaagcaatgtatgcccctcccatcagcggactaattaactgtacatcaaatattacagggctgttattaacaagagatggtggtaaaggaaacaataccaacaccaccgagactttcagacctggaggaggagatatgagggacaattggagaagtgaattatataaatataaaatagtaaaaattgagccattaggggtagcacccaccaaggcaaaaagaagagtggtgcagagagaaaaaagagcagtgacattaggagccatgttccttgggttcttgggagcagcaggaagcactatgggcgcagcgtcagtgacgctracggtacaggccagacaattattgtctggtatagtgcaacagcagaacaatctgctgagggctattgaggcgcaacagcatatgttgcaactcacagtctggggcatcaagcagctccaggcaagagtcctggctgtggaaagatacctaaaggatcaacagctcctagggatttggggttgctctggaaaactcatttgcaccacttctgtgccttggaatgctagttggagtaataaatctctaaatcaaatttgggataatatgacctggatgcagtgggagagggaaattgacaattatacagacataatatacaccttaattgaagaatcgcagaaccaacaagaaaagaatgaactagaattattggaattggataagtgggcaagtttgtggaattggtttgacataacaaattggctgtggtatataaaaatatttataatgatagtaggaggcttagtaggtttaagaatagctttctttgtactttctttagtgaatagagttaggcagggatactcaccattgtcatttcagacccgcctcccaaccctgaggggacccgacaggcccgaaggaaccgaagacgaaggtggagagagagacagagacacatccggacagttagtgactggcttcttcgcactcatctgggtcgatctgcggagcctgtgcctcttcagctaccaccgcttgagagacttactcttgattctagcgaggattgtggaacttctgggacgcagggggtgggagatcctcaaatattggtggaatctcctgcaatattggagtcaggaactaaagaatagtgctgttagtttgcttaatgccacagctatagcagtagctgaggggacagataggattatagaaatagtacaaaggttttttagagctgttctacacatacctagaagaataagacagggcttcgaaagggctttactataaaatgggtggcaagtggtcaaaacgtagtcagaatggatggtctgctgtaagggaaagaatgcacagagctgagccagcagcagagccagcagcagatggggtgggagcagtatctcgag

SEQ ID NO: 5 - DNA, 2698 bp, Artificial Sequence
  Glycoprotein gene for R8.1022
gcggccgcaagaaagagcagaagacagtggcaatgagagtggaggggatcaggaagaattatcagcacttgtggagatggggcaccatgctccttggaatgttaatgatctgtagtgctgcagacaattgtggtcacagtctattatgggtacctgtgtggaaagaagcaaccaccactttattttgtgcctctgatgccaaagcatatgacacagaggtacataatgtttgggccacacatgcctgtgtacccacagaccctaacccacaagaagtagtattggaaaatgtgacagaaaattttaatatggggaaaaataatatggtagatcagatgcatgaggatataatcagtttatgggatcaaagcctaaaaccatgtgtaaaattaaccccactctgtgttactttaaattgcactaatgtgaatgttactaataccaataggaggagtgaaaagatggaaaaaggagaaataaaaaattgctctttccatgtcaccacaagcataaaaagaaaaaaggtgcagaaagaatatgcactttttaataaacttgatgtaatgccaatagataatgaaagctttatattgatacattgtaacaactcaatcattacacaggcttgtccaaaggtatcctttgaaccaattcctatacattattgtgccccggctggttttgcgattctaaagtgtaatgataagaagttcaatggaacaggaccatgtacaaatgtcagtacagtacaatgtacacatggaattaggccagtagtatcaactcaactgctgttaaatggcagtctatcagaaggagaggtagtaattagatctgaaaattttacggacactgttaaaaccataatagtacagctgaatgaatctgtagaaattaattgtacaagacccaacaacaatacaagaaaaggtatacatataggaccagggaaaaatttctatgtaagaagcaaaataataggagatataagacaagcacattgtaacattagtagagcaaaatggaatcacactttagaacagatagttacaaaattaagagaacaatttgggaataaaacaatagtctttaatcaatcctcagggggggacccagaaattgtaatgcacagttttacgtgtggaggggaatttttctactgtaattcaacaaagctgtttagtagtacttggcagtctaataggacttggaaagatactgatgacagtgaaaatatcacactcccatgcagaataaaacaaattgtaaacatgtggcaggaagtaggaaaagcaatgtatgcccctcccatcagtggacgaattagatgttcatcaaatattacagggctgttattaacaagagacggtggtgataccaataacactaacaatgacactgagaccttcagaccgggaggaggaaatatgaaggacaattggagaagtgaattatataaatataaagtagtaaaaattgagccattaggagtagcacccaccaaggcaaagagaagagtggtgcagagagaaaaaagagcagtgggaatgataggagctatgttccttgggttcttgggagcagcaggaagcactatgggcgcagcggccatgacgctgacggtacaggccagactattattgtctggtatagtgcaacagcaaaacaacttgctgagggctattgaggcgcaacagcatctgttgcgactcacagtctggggcatcaagcagctccaggcaagagtcctggctgtggaaagatacctaaaggatcaacagctcctagggatttggggttgctctggaaaactcatctgcaccactgctgtgccttggaatgctagttggagtaataaatctctaaatgaaatttgggataacatgacatggatgcagtgggagagagaaattgaaaattacacaggcttaatatacaacttaattgaacaatcgcagaaccagcaggaaaagaatgaaaaagaattattggaattggataaatggtcaagtttgtggaattggtttagcataacaaactggctgtggtacataaaaatattcataatgatagtaggaggtttaataggtttaagaataattttttctgtactttctttagtgaatagagttaggcagggatactcaccattgtcattccagacccgcctcccagcacagaggggacccgacaggcccgacggaatcgaagaagaaggtggagagagagacagagacaggtccggaccattagtgaatggcttcttagcaatcatctgggtcgatctgcggagcctgttcctcttcagctaccaccgcttgagagacttactcttgattgcagcgaggattgtggaacttctgggacgcagggggtgggaagccctcaaatatctgtggaatctcctgcagtattggagtcaggaactaaagaatagtgctgttagcttgcttaatgtcacggctatagcagtagctgaggggacagatagggttatagaattagcacaaagaattggtaggggtatcctccatatacctagaagaataagacagggctttgaaaggtctatgctataagatgggtgacaagtggtcaaaaagtaagctggggggatggcctgctgtaagagaaagaatgacacgagctgagccacgagctgagccagcagcagatggggtgggagcagtatctcgag

SEQ ID NO: 6 - DNA, 2770 bp, Artificial Sequence
  Glycoprotein gene for R8.1036
gcggccgcaagaaagagcagaagacagtggcaatgagagtgagggagatcaggaagaattatcagcacttgtggaaatggggcaccatgctccttgggatattgatgatctgtagtgctgcagaagaaaatttgtgggtcacagtttattatggggtacctgtgtggaaagaagcaaacaccactttattttgtgcatcagatgctaaagcatattccacagaggcacataatgtttgggccacacatgcctgtgtacccacagaccccagcccacaagaattagtattggaaaatgtgacagaaaattttaacatgtggaaaaataacatggtagaacagatgcatgaggatataatcagtttatgggatcaaagcctaaagccatgtgtaaaattaaccccactctgtgttgctttaaattgcactgatgatttgaggaatgatactgagaacaatagtagtaaagatactattagtccaagaataaagaaaggagaaataaaaaactgctctttcaatatcaccacaaacatgagagataaggtgcagaaacaaaatgcactgttttctaatcttgatgtaatacaaatagataataggacacaaaatagtagtgaaaacaatagtagtaataaatataatagatataagttaataagttgtaatacctcaagagttacacaggcctgtccaaagatatcctttgagccaattcccatacattattgtgccccagctggttttgcgattctaaagtgtaatgataagaagttcaatggaacaggaccatgtaaaaatgtcagcacagtacaatgtacacatggaattaggccagtagtatcaactcaactgctgttaaatggcagtctagcagaaaaagaagtagtaattagatctcaaaatttctcggacaatattaaaaccataatagtacagttgaacgaatctgtagaaattgattgtataagacccaacaacaacacaagaaaaggtatacatatgggaccagggagatattttcatgtaacaggaaatataataggagatataagacaagcacattgtaacattagtagacaaaattggactaacactttggcacagatagctaaaaaattaagagaacaatttgagaatagaacaataaactttactcaacactcaggaggagatccagaaattgtaatgtacacttttaactgtggaggggaatttttctactgtaattcatcacaactgtttaatagtacttggtctaataatactgatgttactaatgttactaagggagagtcagaaactatcacactcccatgtagaataaaacaaattataaacatgtggcaggaagtaggaaaagcaatgtatgcccctcccatcagtggaaaaattagatgtaaatcaaacattacagggctgctattaacaagagatggtgatgttaacataaccaaattcaacaaaaccgagatcttcagacctgaaggaggaaatatgaaggacaattggagaagtgaattatataaatataaagtagtaagaattgaaccattaggaatagcacccaccaaggcaaagagaagagtggtgcagagagaaaaaagagcagtgggaataggagctctgttccttgggttcttgggagcagcaggaagcactatgggcgcagcgtcactaacgctgacggtacaggccagaacattattgtctgatatagtgcaacagcagaacaatttgctgagggctattgaggcgcaacagcatctgttgcaactcacagtctggggcatcaaacagctccaggcaagagtcctggctgtggaaagatacctaagggatcaacagctcctgggaatttggggttgctctggaaaactcatctgcaccactgctgtgccttggaatactagttggagtaataaatctctggattacatttggagtaacatgacctggatgcaatgggaaaaggaaattgacaattacacaggcttaatatataccttacttcaagaatcgcaattccaacaggaaaagaatgaacaagagttattggaattagataaatgggcaagtttgtggaattggtttgatataacaagttggctgtggtatataaaaatattcataatgatagtaggaggcttgataggtttaagaatagttttttctgtattttctatagtaaatagagttaggcagggatattcaccattatcgtttcagacccgcctcccagcacagaggggacccgacaggcccgaaggaatcgaagaagaaggtggagagagagacagagacagatccggtccattagtggatggattcttagcacttatctgggtcgatctgcggagcctgttcctcttcagctaccatcgcttgagagacttactcttgattgtagcgaggattgtggaacttctgggacgcagggggtgggaagccctcaaatattggtggaatctcctgcagtattggagccaggaactaaagaatagtgctgttaacttgcttaatgtcacagccatagcagtagctgagggaacagatagggttctagaaatattacaaagagcttatagagctattatccacatacctagaagaataagacagggcttagaaagggctttgcaataagatgggtggcaagtggtcaaaacgtagtaggagtggatgggatgctataagggaaagaatgagaagaactgggccaggagcaagagctgagccagcagcagatggggtgggagcagtatctcgag

SEQ ID NO: 7 - DNA, 39 bp, Artificial Sequence
  PCR Primer
gaagcggccgcaagaaagagcagaagacagtggcaatga

SEQ ID NO: 8 - DNA, 30 bp, Artificial Sequence
  PCR Primer
gtagcccttccagtccccccttttctttta

SEQ ID NO: 9 - protein, 36 aa, Artificial Sequence
  Synthetic peptide derived from the gp41 region of the HIV-1 envelope glycoprotein
YTSLIHSLIEESQNQQEKNEQELLELDKWASLWNWF

(19) United States
(12) Patent Application Publication
(10) Pub. No.: US 2004/0091853 A1
(43) Pub. Date: May 13, 2004

(54) Viral reporter particles

(76) Inventors: Daria J. Hazuda, Doylestown, PA (US); Janet E. Lineberger, Ambler, PA (US); Michael D. Miller, Chalfont, PA (US); Adam J. Simon, Langhorne, PA (US)

Correspondence Address: MERCK AND CO INC, P O BOX 2000, RAHWAY, NJ, 070650907

(21) Appl. No.: 10/469,199

(22) PCT Filed: Feb. 26, 2002
(86) PCT No.: PCT/US02/05793

(51) Int. Cl. C12Q 1/70; C07H 21/04; C12N 7/00; C07K 14/16; C07H 21/02

(52) U.S. Cl. 435/5; 435/69.3; 435/235.1; 435/320.1; 435/325; 530/395; 536/23.72

(57) ABSTRACT

The present invention features a chimeric protein containing a β-lactamase region and either a Vpr region or a Vpx region. The chimeric protein can be packaged into a viral reporter particle, introduced into a cell recognized by the viral particle and provide intracellular β-lactamase activity.

### CROSS-REFERENCE TO RELATED APPLICATIONS

The present application claims priority to provisional application U.S. Ser. No. 60/272,732, filed Mar. 2, 2001, hereby incorporated by reference herein.

### BACKGROUND OF THE INVENTION

The references cited in the present application are not admitted to be prior art to the claimed invention.

Lentivirus is a viral genus belonging to the retroviridae family. Lentiviruses can be grouped based on the host they infect. Lentiviral groups include the bovine lentivirus group, the equine lentivirus group, the feline lentivirus group, the ovine/caprine lentivirus group, and the primate lentivirus group. The primate lentivirus group is further divided into human immunodeficiency virus 1 (HIV-1), human immunodeficiency virus 2 (HIV-2), and simian immunodeficiency virus (SIV). (Virus Taxonomy, van Regenmortel et al., (eds.) Academic Press, San Diego, Calif. 2000.)

The lentiviral genome contains structural and accessory genes flanked by 3′ and 5′ long terminal repeat (LTR) sequences. LTR sequences contain regions important for expression and processing of the encoded polypeptides. (Field's Virology, Fields et al., (eds.) 3d edition. Lippincott-Raven Publishers, Philadelphia, Pa. 1996.)

Lentiviral structural genes are gag, pol, and env. These genes encode different precursor polyproteins. The Gag precursor (Pr55^{gag}) is processed into the matrix, capsid, nucleocapsid, and p6. The Pol precursor is processed into protease, reverse transcriptase and integrase. The Env precursor is processed to form glycoproteins.

The Gag precursor and its proteolytic cleavage products are the major structural components of the lentiviral virion. Accumulation of Gag proteins at the plasma membrane leads to the assembly of immature virions that bud from the cell surface. Inside the nascent virion, Pr55^{gag} as is cleaved by a protease into the matrix, capsid, nucleocapsid and C-terminal p6 domain. Gag processing causes a reorganization of the internal virion structure. (Weigers et al., J. Virology 72:2846-2854, 1998.)

Pr55^{gag} facilitates virion incorporation of the accessory proteins Vpx and Vpr. The HIV-1 C-terminal p6 domain facilitates virion incorporation of Vpr. (Lavallée et al., J. Virol. 68:1926-1934, 1994, Paxton et al., J. Virol. 67:7229-7237, 1993, Lu et al., J. Virol. 67:6542-6550, 1993.) Similarly, the C-terminal region of the HIV-2 Gag polyprotein precursor facilitates incorporation of HIV-2 Vpx. (Wu et al., J. Virol. 68:6161-6169, 1994.)

Vpx and Vpr have been used as components of chimeric proteins. (Wu et al,. J. Virol. 69:3389-3398, 1995, Wu et al., EMBO Journal 16:5113-5122, 1997, Cohen et al., U.S. Pat. No. 5,861,161, Sato et al., Microbiol. Immunol. 39:1015-1019, 1995, Kobinger et al., J. Virology 72:5441-5448, 1998, Yao et al., Gene Therapy 6:1590-1599, 1999, Liu et al, J. Virol. 71:7704-7710, 1997, Stauber et al., Biochemical and Biophysical Research Communications 258:695-702, 1999.)

### SUMMARY OF THE INVENTION

The present invention features a chimeric protein containing a β-lactamase region and either a Vpr region or a Vpx region. The chimeric protein can be packaged into a viral reporter particle, introduced into a cell recognized by the viral particle and provide intracellular β-lactamase activity.

Both the orientation of the Vpr/Vpx region to the β-lactamase region and the presence of HIV protease sites between the regions were found to affect production of intracellular β-lactamase activity. Preferred constructs contained the Vpr/vpx region carboxy to the β-lactamase region. In addition, HIV protease sites resulting in intracellular cleavage of a Vpr region from a β-lactamase region decreased β-lactamase activity. More preferred constructs lack HIV protease sites between the Vpr/Vpx region and the β-lactamase region.

Viral reporter particles described herein are based on a lentiviral virion, preferably an HIV virion. The virion contains viral components needed for the incorporation of β-lactarnase-Vpr/Vpx chimeric proteins and the production of an entry competent virion.

A “entry competent virion” is a virion containing a β-lactamase-Vpr/Vpx chimeric protein that interacts with a target cell in a manner allowing entry of the chimeric protein into the cell. Entry is mediated by one or more virion envelope glycoproteins that recognize one or more receptors present on a target cell.

A viral reporter particle may contain virion components including envelope glycoproteins from a particular lentivirus such as HIV-1 or HIV-2. Alternatively, the viral reporter particle can be pseudotyped with envelope glycoproteins from a virus outside of the lentiviral genus.

Thus, a first aspect of the present invention describes a chimeric protein comprising a β-lactamase region and a Vpr or Vpx region. The Vpr or Vpx region is on the carboxy side of the β-lactamase region. The chimeric protein can be packaged in an entry competent lenti virus particle and has β-lactamase activity.

The Vpr/Vpx region can target the chimeric protein into a viral reporter particle such as a naturally occurring lentiviral particle, preferably an HIV particle. The ability to be packaged into a lentiviral particle such as HIV does not exclude the ability to be packaged into other particles such as pseudotyped HIV particles.

Another aspect of the present invention describes an expression vector comprising nucleic acid expressing a chimeric β-lactamase-Vpr/Vpx protein. Reference to “expressing” a protein indicates the presence of regulatory elements providing for the functional expression of the protein inside a cell.

Regulatory elements needed for the functional expression of a protein are well known in the art. Such elements include a promoter and a ribosome binding site. Additional elements that may be present include an operator, enhancer and a polyadenylation region.

Another aspect of the present invention describes an entry competent viral reporter particle containing a chimeric β-lactamase-Vpr/Vpx protein. The particle also contains (a) one or more viral envelope glycoproteins, (b) a lipid bilayer, (c) an HIV matrix capsid, (d) an HIV capsid, (e) an HIV nucleocapsid, and (f) an HIV C-terminal p6 domain.

Another aspect of the present invention describes an entry competent viral reporter particle made by a process comprising the steps of: (a) cotransfecting a cell with one or more nucleic acids that together express a β-lactamase-Vpr/Vpx chimeric protein and components needed to produce an entry competent viral reporter particle containing one or more envelope glycoproteins; and (b) growing the cell cotransfected in step (a) under viral production conditions to produce the viral particle. The β-lactamase-Vpr/Vpx chimeric protein is packaged by the viral reporter particle and has β-lactamase activity.

Another aspect of the present invention describes a method of measuring the ability of a compound to inhibit viral entry into a cell. The method involves the steps of: (a) combining together (i) an entry competent viral reporter particle comprising a β-lactamase-Vpr/Vpx chimeric protein having β-lactamase activity, (ii) a target cell, and (iii) the compound, under conditions allowing entry of the viral particle into the target cell in the absence of the compound; and (b) measuring β-lactamase activity in the host cell as a measure of the ability of the compound to inhibit viral entry.

Another aspect of the present invention describes a method of measuring the ability of a compound to inhibit mature virus production. The method involves the steps of: (a) growing a recombinant cell able to produce a viral particle comprising a β-lactamase-Vpr/Vpx chimeric protein under viral production conditions in the presence of the compound, and (b) measuring the production of entry competent viruses that can provide β-lactamase activity to a cell as an indication of the ability of the compound to inhibit mature virus production. Viral production conditions are conditions compatible with the production of a virion.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 illustrates the ability of a HIV based viral reporter particle assay to provide β-lactamase activity to a cell.

FIG. 2 depicts the plasmid pMM310 encoding a fusion protein consisting of a bacterial β-lactamase enzyme fused to the HIV accessory protein Vpr.

FIG. 3 shows that the specific HIV entry inhibitor DP-178 blocks HIV reporter particle mediated transfer of β-lactamase to target cells. HIV reporter particles were incubated with target cells for 5 hours at 37° C. in the presence of various concentrations of the peptide inhibitor DP-178 and then loaded with the fluorescent β-lactamase substrate CCF2-AM. The graph shows blue fluorescence emissions (y axis) as a function of DP-178 concentration (x axis). Two different HIV reporter particles were tested, one generated from the R8 HIV provirus and one generated from the R8.BaL provirus.

FIG. 4 shows that the specific HIV entry inhibitor IgGlb12 blocks the HIV reporter particle mediated transfer of β-lactamase to target cells. HIV reporter particles were incubated with target cells for 5 hours at 37° C. in the presence of various concentrations of the antibody IgGlb12 and then loaded with the fluorescent β-lactamase substrate CCF2-AM. The graph shows blue fluorescence emissions (y axis) as a function of IgGlb12 concentration (x axis). Two different HIV reporter particles were tested, one generated from the R8 HIV provirus and one generated from the R8.BaL provirus.

FIG. 5 shows a graph of blue fluorescence emission (y axis) from CCF2-AM-loaded SupT1 cells as a function of input HIV reporter particle. Prior to loading with CCF2-AM, cells were incubated with dilutions of HIV reporter particle bearing no envelope glycoprotein, the vesicular stomatitis virus G envelope glycoprotein, or the amphotropic murine leukemia virus envelope glycoprotein.

FIG. 6 shows a graph of blue fluorescence emission (y axis) from CCF2-AM-loaded SupT1 cells as a function of input HIV reporter particle. Prior to loading with CCF2-AM, cells were incubated with dilutions of HWV reporter particle produced from 293T cells transfected with various reagents: CaPO₄, Fugene6, Effectene, or TransIT.

### DETAILED DESCRIPTION OF THE INVENTION

Chimeric β-lactamase-Vpr/Vpx proteins provide a useful reporter for assays measuring the production of an entry competent virion and the ability of the virion to infect a cell. Such assays have different applications including being used as a tool for basic research, as a tool for obtaining antiviral compounds, and as a tool for evaluating antiviral compounds. Basic research applications include further studying the production of viruses and viral interaction with a cell.

Obtaining and evaluating antiviral compounds have therapeutic implications. Compounds inhibiting the formation of a virion or the ability of the virion to infect a cell may be useful for therapeutic antiviral treatment. Such treatment can be directed to a patient having a viral infection or can be a prophylactic treatment. Treatment of a patient with a disease alleviates or retards the progression of the disease. A prophylactic treatment reduces the likelihood or severity of a disease.

Chimeric β-lactamase-Vpr/Vpx proteins

Chimeric β-lactamase-Vpr/Vpx have two components (1) a β-lactamase region providing detectable enzymatic activity and (2) a Vpr or Vpx region that targets the protein to a virion. β-lactamase-Vpr/Vpx protein have the proper size for integration into a virion in sufficient numbers to provide detectable intracellular β-lactamase activity upon host entry.

The Vpr/Vpx and β-lactamase regions can be directly joined to each other or can be joined together by a polypeptide linker. A preferred orientation has the Vpr/Vpx region on the carboxy side of the β-lactamase region.

If present, the size and sequence of the polypeptide linker should be chosen so as not to substantially affect the ability of a particular β-lactamase-Vpr/Vpx protein to packaged inside a virion and possess intracellular β-lactamase activity. In different embodiments, a linker is between about 2 to about 50 amino acids, about 2 to about 20 amino acids, about 2 to about 10 amino acids, and about 2 amino acids. Preferably, the linker does not contain any HIV protease recognition sequences.

Vpr/Vpx Region

A chimeric β-lactamase-Vpr/Vpx protein contains a sufficient Vpr or Vpx region for virion packaging. In a preferred embodiment, a Vpr region from HIV is present.

Vpr is generally present in primate lentiviruses including HIV-1 and is incorporated in trans into a viral particle. A Vpr region present in a β-lactamase-Vpr chimeric protein is capable of interacting with a Gag polyprotein precursor such that it can be packaged by an lentivirus virion, preferably, a HIV-1 virion. The ability to be packaged by an HIV virion does not exclude the ability to be packaged by other types of virions.

Suitable Vpr regions include naturally occurring Vpr regions and functional derivatives thereof able to interact with the Gag polyprotein precursor. The affect of different alterations to naturally occurring Vpr on its ability to interact with the Gag polyprotein precursor and be packaged by a virion is well known in the art. (See, for example, Paxton et al., J. Virol. 67:6542-6550, 1993, Yao et al., Gene Therapy, 6:1590-1599, 1996, Sato et al., Microbiol. Immunol 39:1015-1019, 1995, Cohen et al., U.S. Pat. No 5,861,161.) Preferably, the Vpr region that is present contains the N-terminal α-helix region.

Vpx is present in HIV-2. The importance of different Vpx amino acids or regions on the ability of Vpx to be packaged by a virion are well known in the art. (See, for example, Sato et al., Microbiol. Immunol 39:1015-1019, 1995, and Cohen et al., U.S. Pat. No 5,861,161). Preferably, the Vpx region that is present contains the N-terminal α-helix region.

β-lactamase

The β-lactamase region provides detectable intracellular β-lactamase activity. β-lactamase activity catalyzes the cleavage of the β-lactam ring present in cephalosporins.

The β-lactamase region can be provided, for example, from β-lactamases well known in the art and functional derivatives thereof. References such as Ambler, Phil. Trans R. Soc. Lond. Ser. B. 289:321-331, 1980, provide examples of naturally occurring β-lactamases.

Functional Derivatives

Functional derivatives can be produced by altering a naturally occurring sequence. Examples of common alterations include substitutions, deletions, and additions of amino acids or amino acid regions. Functional derivatives can be produced by modifying a nucleic acid sequence encoding for a naturally occurring sequence and expressing the modified nucleic acid. Recombinant techniques for producing and purifying proteins are well known in the art. (For example, see, Ausubel, Current Protocols in Molecular Biology, John Wiley, 1987-1998, and Sambrook, et al., Molecular Cloning, A Laboratory Manual, 2^{nd} Edition, Cold Spring Harbor Laboratory Press, 1989.)

One method of designing altered proteins is to take into account amino acid R-groups. An amino acid R group affects different properties of the amino acid such as physical size, charge, and hydrophobicity. Amino acids can be divided into different groups as follows: neutral and hydrophobic (alanine, valine, leucine, isoleucine, proline, tryptophan, phenylalanine, and methionine); neutral and polar (glycine, serine, threonine, tyrosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolor amino acids in the interior of a polypeptide then glutamate because of its long aliphatic side chain. (See, Ausubel, Current Protocols in Molecular Biology, John Wiley, 1987-1998, Supplement 33 Appendix IC.) Derivatives can also be produced to enhance intracellular activity. An example of such a derivative is TEM-1 β-lactamase. (Kadonaga et al., J. Biol. Chem. 259:2149-2154, 1984.) TEM-1 β-lactamase is a derivative of E. coli β-lactamase, where the signal sequence is deleted. The deletion of the signal sequence increases cytoplasmic accumulation.

Polypeptide Production

A β-lactamase-Vpr/Vpx chimeric protein can be produced by recombinant means using nucleic acid encoding the protein. Nucleic acid encoding a chimeric protein can be inserted into a host genome or can be part of an expression vector.

Preferably, an expression vector is used to produce the β-lactamase-Vpr/Vpx chimeric protein. An expression vector contains nucleic acid encoding a polypeptide along with regulatory elements for proper transcription and processing. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Starting with a particular amino acid sequence and the known degeneracy of the genetic code, a large number of different encoding nucleic acid sequences can be obtained. The degeneracy of the genetic code arises because almost all amino acids are encoded by different combinations of nucleotide triplets or “codons”. The translation of a particular codon into a particular amino acid is well known in the art (see, e.g., Lewin, GENES IV, p. 119, Oxford University Press, 1990). Amino acids are encoded by codons as follows:

A = Ala = Alanine:
codons GCA, GCC, GCG, GCU;
C = Cys = Cysteine:
codons UGC, UGU;
D = Asp = Aspartic acid:
codons GAC, GAU;
E = Glu = Glutamic acid:
codons GAA, GAG;
F = Phe = Phenylalanine:
codons UUC, UUU;
G = Gly = Glycine:
codons GGA, GGC, GGG, GGU;
H = His = Histidine:
codons GAG, CAU;
I = Ile = Isoleucine:
codons AUA, AUG, AUU;
K = Lys = Lysine:
codons AAA, AAG;
L = Leu = Leucine:
codons UUA, UUG, CUA, CUC, CUG, CUU;
M = Met = Methionine:
codon AUG;
N = Asn = Asparagine:
codons AAC, AAU;
P = Pro = Proline:
codons CCA, CCC, CCG, CCU;
Q = Gln = Glutamine:
codons CAA, CAG;
R = Arg = Arginine:
codons AGA, AGG, CGA, CGC, CGG, CGU;
S = Ser = Serine:
codons AGC, AGU, UCA, UCC, UCG, UCU;
T = Thr = Threonine:
codons ACA, ACC, ACG, ACU;
V = Val = Valine:
codons GUA, GUC, GUG, GUU;
W = Trp = Tryptophan;
and codon UGG;
Y = Tyr = Tyrosine:
codons UAC, UAU.

Viral Reporter Particle

Reporter particles can recognize a target cell and deliver a β-lactamase-Vpr/Vpx chimeric protein into the cell. Target cell recognition is achieved by particle glycoproteins. Reporter particles can be produced with glycoproteins naturally associated with other viral components that are present. Reporter particles can also be pseudotyped to contain glycoproteins not naturally associated with other viral components that are present.

Production of viral particles in a host cell is mediated by the Gag polyprotein. The resulting particle is produced by viral budding at the plasma membrane and contains a lipid bilayer incorporating glycoproteins. The incorporated glycoproteins determine the host specificity of the viral particle.

Preferably, the reporter particle is an HIV particle containing a β-lactamase-Vpr/Vpx chimeric protein, one or more viral envelope glycoproteins, a lipid bilayer, an HIV matrix capsid, an HIV capsid, an HIV nucleocapsid, and an HIV C-terminal p6 domain. Different types of viral envelope proteins may be present affecting the cell specificity of the viral particle.

Reference to HIV components present in a viral particle indicates naturally occurring components or functional derivatives thereof. Functional derivatives are based on a naturally occurring sequence containing one or more alterations not substantially affecting formation of the viral particle or the ability of the viral particle to infect a cell. The ability of a derivative to package a β-lactamase-Vpr/Vpx chimeric protein and infect or enter a cell can be evaluated using techniques such as those described in the Examples provided below.

Sequence variations for HIV viral components are well known in the art. The different variations provide examples of different sequences that can serve as HIV viral components and as starting points for producing functional derivatives.

Viral envelope glycoproteins that may be present include those from different lentivirus and those from other types of viruses. Preferred lentivirus glycoproteins are HIV gp120 and HIV gp41. HIV envelope glycoproteins target different cell types such as primary cultures of monocyte-derived macrophages and T lymphoid cells and certain transformed cell lines. In different embodiments the HIV gp120 is CCR5 tropic, examples of which include HIV gp 120 from HIV Bal, JRFL, SF162, and YU2; and the HIV gp120 is CXCR4 tropic, examples of which include HIV gp120 from HIV NLA-3, R8 and MN.

Viral envelope glycoproteins present from a non-lentivirus that may be present include those from vesicular stomatitis virus (VSV), amphotropic murine leukemia virus (AMLV), and hepatitis C virus (HCV). VSV glycoprotein targets a large number of cells including primary chick embryo cells, BHK-21 cells, Vero cells, mouse L cells and Chinese hamster ovary cells. (Field's Virology, Fields et al., (eds.) 2^{nd} edition. New York, Raven Press, 1990.) AMLV glycoprotein target cells such as NU3T3 cells (mouse fibroblasts), A431 cells (human keratinocytes), and H9 cells (human T cells). (Bachrach et al., J. Virol. 74:8480-8486, 2000). HCV E1 and E2 target cells such as HepG2, Huh7, and FLC4. (Takikawa et al., J. Virol., 74:5066-5074,2000).

Pseudotyping can be carried out using a complete glycoprotein from a non-lentivirus or with a chimeric protein containing a glycoprotein region with a lentivirus region and a non-lentivirus region. For example, pseudotyping a HIV virion with VSV envelope glycoprotein can be achieved with a complete VSV envelope glycoprotein, or a chimeric VSV envelope glycoprotein containing the extracellular VSV envelope glycoprotein domain fused to transmembrane HIV envelope glycoprotein.

Viral Reporter Particle Production

Viral reporter particles can be produced by expressing nucleic acid encoding a β-lactamase-Vpr/Vpx chimeric protein in combination with nucleic acid encoding viral components needed for the production of an entry component virion. The reporter particle can also contain additional components such as nucleic acid encoding one or more additional lentivirus, preferably, HIV genes.

Additional components that are present need not be functional. In a preferred embodiment, the viral reporter particle is entry competent and replication incompetent. A replication incompetent viral reporter particle can be produced in different ways such as eliminating or altering one or more genes needed for viral replication. Replication incompetent viral reporter particles offer safety advantages over viral reporter particles able to replicate. lentivirus vectors have attracted interest as vectors for gene therapy. (For example, see Dull et al., J. Virol. 72:8463-8471, 1988, and Naldini et al., Science 272:263-267, 1996.) Based on the guidance provided herein techniques for producing lentivirus vectors can be modified to produce a viral reporter particle incorporating a β-lactamase-Vpr/Vpx Chimeric Protein.

Modifications to techniques for producing lentivirus vectors such that a viral reporter particle is produced take into account incorporation of the β-lactamase-Vpr/Vpx chimeric protein and the use of desired envelope proteins. Incorporation of β-lactamase-Vpr/Vpx chimeric protein occurs in trans by interaction with the Gag precursor. Thus, nucleic acid encoding a β-lactamase-Vpr/Vpx chimeric protein need not be part of nucleic acid encoding for other viral components.

Nucleic acid encoding different viral components can be introduced and expressed in a cell by altering the host genome or through the use of expression vectors. Alteration of the host genome involves introducing nucleic acid into the genome such that the nucleic acid is expressed. Preferably, nucleic acids encoding viral components are provided on one or more expression vectors.

Viral reporter particles can be produced in transformed human cells. An example of a suitable cell type is HEK-293.

β-lactamase Assays

Intracellular β-lactamase activity is preferably measured using a fluorogenic substrate that is cleaved by β-lactamase. Preferred substrates are membrane permeant fluorogenic substrates that become membrane impermeant inside a cell, and that are cleaved by β-lactamase to produce a detectable signal. Examples of such substrates are provided in Zlokarnik et al., Science 279:84-88, 1998, and Tsien et al., U.S. Pat. No. 5,741,657.

In an embodiment of the present invention, a cell-permeant fluorescent β-lactamase substrate such as CCF2-AM or CCF4-AM (Aurora Biosciences, Inc., San Diego, Calif.) is loaded into a cell. These substrates contain an ester group facilitating transport across the cell membrane. Inside the cell, the ester group is cleaved rendering the substrate membrane impermeant. The intact substrates, when stimulated with light of ˜405 nm, emit green fluorescence (530 nm) due to resonant energy transfer from a coumarin to fluorescein dye molecule. Upon cleavage of the substrates by β-lactamase, the fluorescence emission changes to a blue color (˜460 nm) of only the coumarin. The fluorescence emissions of the substrate present in the cells can be detected by, for example, fluorescence microscopy or by a fluorometer in conjunction with appropriate emission and excitation filters.

Entry Inhibition and Viral Formation Assays

β-lactamase-Vpr/Vpx chimeric protein can be used in assays measuring the production and activity of viral reporter particles. Such assays can be used to identify viral inhibitors, such as inhibitors of HIV, HCV, AMLV, and VSV. Antiviral compounds can be used in vitro or in vivo.

Measuring the ability of a compound to inhibit viral entry into a cell can be performed by combining together an entry competent viral reporter particle comprising a β-lactamase-Vpr/Vpx chimeric protein, a compatible target cell, and a test compound. The assay is performed under conditions allowing entry of the viral particle into the host cell in the absence of the compound. In an embodiment of the present invention, the target cell is a primary human cell.

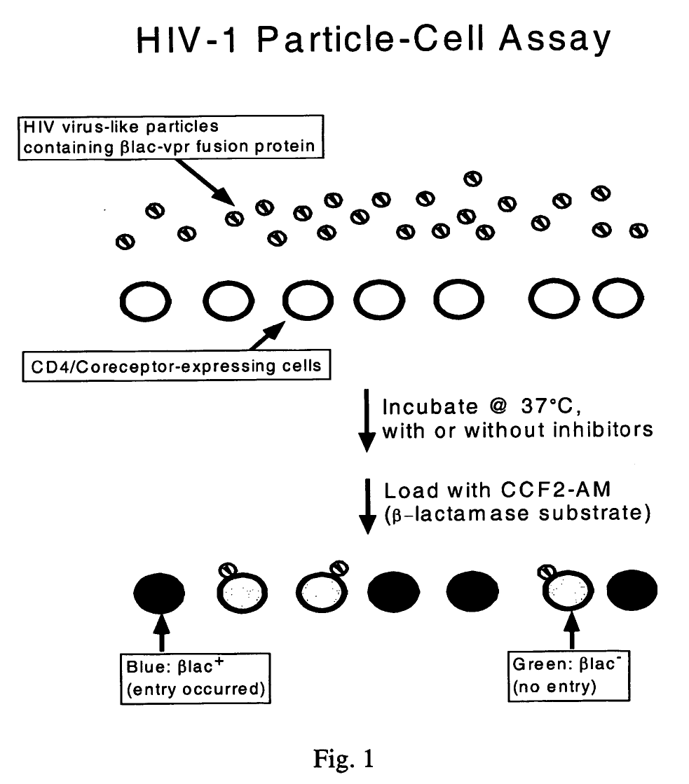

FIG. 1 illustrates an example of a viral inhibition assay using HIV-1 reporter particles. The ability of the compound to inhibit viral entry is evaluated by observing β-lactamase activity.

Entry inhibition assays can be performed using pseudotyped viral particles to identify inhibitors of different types of viruses. For example, viral particles containing gp41 and gp120 can be used to assay for HIV entry inhibitors, and HCV E1 and E2 pseudotyped viral particles can be used to assay for HIV entry inhibitors.

Measuring the ability of a compound to inhibit mature virus production can be performed by growing a recombinant cell able to produce a viral reporter particle comprising a β-lactamase-Vpr/Vpx chimeric protein under viral production conditions in the presence of a test compound. The ability of the test compound to inhibit viral production is determined by evaluating the production of virions able to provide β-lactamase to a host cell. If desired, a mature virus inhibition assay can be performed using pseudotyped viral particles to alter target cell specificity.

### EXAMPLES

Examples are provided below to further illustrate different features of the present invention. The examples also illustrate useful methodology for practicing the invention. These examples do not limit the claimed invention.

Example 1: Material and Methods

This example illustrates some of the material and methods employed to produce and evaluate viral reporter particles.

Plasmid DNA

Plasmids were constructed, fermented and purified using standard recombinant nucleic acid techniques.

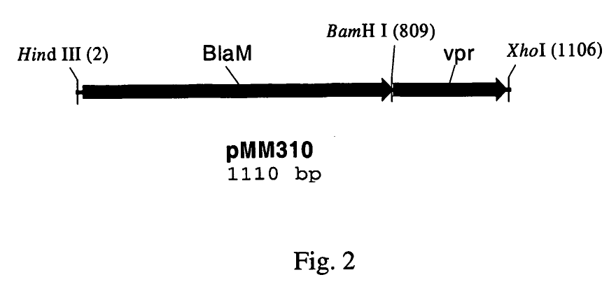

pMM3 10 (FIG. 2) encodes a fusion protein consisting of the bacterial β-lactamase gene (designated BlaM, from Aurora Biosciences, Inc.) to vpr of HIV-1 (strain YU2; Li et al., J. Virol. 66:6587, 1992). The BlaM-vpr fusion sequence is cloned into the HindIII and XhoI sites of the vector pcDNA3.1/zeo(+) (from Invitrogen, Carlsbad, Calif.). The nucleotide sequence of the β-lactamase-Vpr construct is displayed in SEQ. ID. NO. 1. The amino acid sequence encoded by this construct is displayed in SEQ. ID. NO. 2.

pMM304 contains an HIV proviral DNA derived from strain YU2 (Li et al., J. Virol. 66:6587, 1992) by removal of a restriction digestion fragment. Plasmid pYU2 was digested with Pacd (nt6190) and BsaBI (nt7521), the ends were made blunt using T4 DNA polymerase, and the plasmid was recircularized using T4 DNA ligase. (Li et al., J. Virol. 66:6587, 1992). The resulting plasmid contains a genetic deletion such that the envelope glycoprotein gene is not expressed.

pMM312 contains an HIV proviral DNA derived from pMM304 by removal of a 2.6kb fragment restriction digestion fragment. Plasmid pMM304 was digested with BstEII (nt3011) and NcoI (nt5665), the ends were made blunt using the Klenow fragment of E. coli DNA polymerase I, and the plasmid was recircularized using T4 DNA ligase. The resulting proviral DNA lacks intact sequences coding for reverse transcriptase, integrase, vif, vpr, and envelope.

pNLA-³ represents a canonical wild-type HIV provirus. (Adachi et al., J. Virol. 59:284-291, 1986; Salminen et al., Virology 213:80-86, 1995; GENBANK accession U26942.)

pRL500 is a derivative of pNlA-3 containing mutations in the integrase coding sequence such that the integrase protein contains 2 amino acid sequence changes. The changes, vall51 changed to glu and aspl52 changed to g/n, render the integrase enzyme defective such that viruses produced from pRL500 are replication incompetent. (LaFemina et al., J. Virol. 66:7414-7419, 1992.)

R8 (Gallay et al., J. Virol. 70:1027-1032, 1996; obtained from C. Aiken, Vanderbilt U., Nashville, Tenn.) contains a hybrid HIV provirus, part of which is derived from the pNIA-3 sequence and part of which is derived from another canonical wild-type HIV strain, HXB2. (Ratner et al., AIDS Res. Hum. Retroviruses 3:57, 1986.)

R8.Bal is a derivative of R8 in which most of the envelope gene has been replaced by the corresponding envelope gene of the HIV-1 primary isolate BaL. (Gallay et al., J. Virol. 70:1027-1032, 1996; obtained from C. Aiken, Vanderbilt U., Nashville, Tenn.).

R9 PR Δenv represents a derivative of R8 in which genetic deletions have been introduced into the protease (PR) and envelope (env) genes. These deletions prevent expression of functional PR and env proteins. (Wymna et al., J. Virol., 74:9381-9387, 2000; obtained from C. Aiken, Vanderbilt U., Nashville, Tenn.)

pYU2 contains an HIV provirus from the YU2 isolate of HIV. (Li et al., J. Virol. 66:6587, 1992; GENBANK accession #M93258; obtained from the AIDS Research and Reference Reagent Program, Bethesda, Md.).

pCMV-VSVG contains the envelope glycoprotein sequence from the VSV under the control of the cytomegalovirus early promoter (obtained from J. Kappes, University of Alabama at Birmingham). (Wu et al., J. Virol. 73:2126-2135, 1999; Liu etal, J. Virol. 73:8831-8836, 1999.)

pSV-A-MLV contains the sequence encoding the AMLV envelope glycoprotein. (Landau et al., J. Virol 65:162, 1991; obtained the AIDS Research and Reference Reagent Program, Bethesda, Md.).

pMM326 is a derivative of R8 in which a unique NotI restriction enzyme site has been inserted upstream of the envelope gene. This enzyme site allows insertion of gp160 genes cloned from other HIV isolates. The nucleotide sequence of the modified proviral DNA is presented as SEQ. ID. NO. 3.

Plasmids pR8.1021, pR8.1022, and pR8.1036, represent derivatives of plasmid pMM326 into which have been cloned the envelope glycoprotein genes of primary HIV isolates 1021, 1022, and 1036, respectively. The derivatives contain a cloned glycoprotein gene replacing bases 6314-9017 (encoding endogenous envelope glycoprotein) in SEQ. ID. NO. 3. The nucleotide sequences of the envelope glycoprotein genes from R8.1021, R8.1022, and R8.1036 are presented as SEQ. ID. NO. 4, SEQ. ID. NO. 5, and SEQ. ID. NO. 6, respectively.

Oligonucleotides

Synthetic oligonucleotides were supplied by Midland Certified Reagent Company (Midland, Tex.).

Oligo MM439 (SEQ. ID. NO. 7: 5′GAAGCGGCCGCAAGAAAGAGCAGAAG ACAGTGGCAATGA-3′) represents the envB oligonucleotide (described in Gao et al., J. Virol. 70:651-1667, 1996) to which a NotI sequence (underlined) and some additional nucleotides were appended at the 5 ′ end to facilitate cloning of PCR products.

Oligo MM440 (SEQ. ID. NO. 8: 5′GTAGCCCTTCCAGTCCCCCCTTTTCTTTA-3′) represents the envM oligonucleotide (described in Gao et al., J. Virol. 70:651-1667, 1996) to which a single G residue was added at the 5 ′ end.

Cells

Transformed cell lines and primary cells described below were prepared and cultured by standard methods familiar to those skilled in the art. 293T cells are derivatives of HEK293, transformed human embryonic kidney cells, which have been engineered to express the SV40 large T antigen. The cells are maintained in Dulbecco's Modified Eagle's Medium (DMEM; Lifetechnologies, Gaithersberg, Md., Cat. #11960-044 supplemented with 10% fetal bovine serum (FBS; Lifetechnologies or Hyclone, Logan, Utah). For virus production after transfection, cells are maintained in DMEM lacking phenol red (Lifetechnologies, Cat. #21063-029) and supplemented with 10% fetal bovine serum.

SupT1 cells are a transformed human T cell line. SupT1 cells were maintained in RPMI 1640 (Lifetechnologies, Cat. #11875-093) supplemented with 10% FBS. In some cases, derivatives of SupT1 cells were transfected to stably express the human CCR5 gene. CCR5-expressing SupT1 cells were maintained in RPMI 1640/10% FBS containing 0.4 μg/ml Puromycin (Clontech, Palo Alto, Calif.).

Peripheral blood mononuclear cells (PBMCs) were isolated from human blood by standard techniques known to those skilled in the art (Ficoll/Hypaque density centrifugation) and maintained in RPMI1640/10% FBS.

Human monocyte-derived macrophages were obtained from human PBMCs. PBMCs were plated in plastic flasks for >20 minutes to allow monocyte adherence, and non-adherent cells were removed by washing. Monocytes were detached from the plastic with Versene (Cellgro, Herndon, Va.), washed, resuspended at 10⁶cells/ml in monocyte/macrophage culture medium (DMEM, 10% FBS, 10% horse serum, 20 ng/ml each M-CSF an GM-CSF [both from R&D Systems (Minneapolis, Minn.)]) and cultured in Teflon jars at 37° C./5% CO₂ for 72 hours. The medium was then replaced and cells were cultured an additional 72 hours before use in assays.

Assay Reagents

Fugene6 is a lipidic transfection reagent supplied commercially by Roche (Cat. #1815091). OptiMEM is a serum-free medium supplied by LifeTechnologies (Cat. #31985-070). These reagents are used together to generate HIV viral particles by transfecting cells with plasmid DNA.

Reagents enabling transfection of cells with DNA by means of a calcium phosphate-DNA precipitate were purchased from Promega Corp. (Madison, Wis., Profection calcium phosphate kit, Cat. # E1200).

CCF2-AM and CCF4-AM are cell-permeant fluorescent substrates for the enzyme β-lactamase and are commercially available from Aurora Biosciences, Inc. (San Diego, Calif.). These reagents are used in conjunction with two “cell-loading” solutions (solutions B and C) also supplied by Aurora.

Indinavir (Merck & Co., Inc., Rahway, N.J.) is an HIV protease inhibitor, which blocks virion maturation and infectivity.

DP-178 is a synthetic peptide derived from the gp41 region of the HIV-1 envelope glycoprotein. DP-178 inhibits the entry of HIV-1 virions driven by the HIV-1 envelope glycoprotein. The amino acid sequence of DP-178 is acetyl-YTSLIHSLIEESQNQQEKNEQELLELDKWASLWNWF-amide (SEQ. ID. NO. 9). (Wild et al., Proc. Natl. Acad. Sci. USA, 91:9770-9774, 1994.)

IgGlb12 is a humanized immunoglobulin reactive to HIV-1 envelope glycoprotein gp120 derived from certain HIV strains. (Burton et al., Science 266:1024-1027, 1994.) IgGlb12 can block HIV-1 infectivity.

Expand high-fidelity PCR system was from Roche (Cat. #1732641).

Effectene is a commercially available transfection reagent (Qiagen, Inc., Valencia, Calif., Cat. #301425.)

TransIT is a commercially available transfection reagent (Panvera Corp., Madison, Wis., Cat. #M1R2300).

L-697661 (Merck & Co., Inc., Rahway, N.J.) is a non-nucleoside reverse transcriptase inhibitor that inhibits synthesis of HIV cDNA in newly infected cells. (Goldman et al., Proc. Natl. Acad. Sci. USA. 88(15):6863-6867, 1991.)

Instruments

Cells loaded with the fluorescent β-lactamase substrate CCF2-AM or CCF4-AM were viewed by epifluorescence microscopy using an Olympus IX70 inverted microscope equipped with a mercury vapor lamp and the β-lactamase filter set from Chroma Technologies (Battleboro, Vt., Cat. #41031).

Blue and green fluorescence in cells loaded with CCF2-AM or CCF4AM were quantified using a PolarStar fluorometer (BMG, Durham, N.C.) equipped with a 410±12 nm excitation filter (Chroma Catalog #020410-12), a 460±10 nm emission filter (Chroma Catalog #020-460-10), and a 530±12 nm emission filter (Chroma Catalog #020-530-12).

Example 2: HIV Virions Pseudotyped with VSV-G

This example illustrates the production and use of a viral particle based on a HIV virion that is pseudotyped with the envelope glycoprotein VSV-G. The reporter particle was able to deliver enzymatically active β-lactamase to a target cell.

VSV-G Pseudotyped Reporter Particle

H1V virions carrying a β-lactamase-Vpr chimeric protein and bearing the promiscuous envelope glycoprotein VSV-G were generated by cotransfecting 293T cells with plasmid DNAs pMM304 ( proviral DNA lacking a functional envelope gene), pMM310 (β-lactamase-vpr fusion) and pCMV-VSVG by the calcium phosphate method (Promega Profection CaPO4 transfection kit). For transfections, a confluent flask of 293T cells was treated with trypsin/EDTA solution to remove cells, and 1/50 of the cells were plated into each well of a 6-well plate. The following day, cells were transfected with DNA mixes as follows:

Well 1: 0.5 μg pMM304, 1 μg pMM310, 0.5 μg pcDNA3.1

Well 2: 0.5 μg pMM304, 1 μg pMM310, 0.5 μg pCMV-VSVG

Well 3: 0.5 μg pMM304, 1 μg pMM310, 0.25 μg HXB2 gp160, 0.1 μg pRSV-rev

For transfection, each DNA mixture (˜2 μg total) was diluted into 44 μl H₂O and then 6 μl of 2.5 M CaCl₂ (from kit) were added. Each solution was added dropwise to 150 μl of HEPES-buffered saline solution (from kit) with vigorous agitation, incubated at room temperature for 30 minutes, and then added dropwise to one well of 293T cells. Cells were incubated at 37° C./5% CO₂. Three days later, culture supernatants were harvested and brought to 20 mM HEPES by addition of a 1 M HEPES solution, pH 7.3. Supernatants were tested by incubating 90 μl of each supernatant with 10 μl of SupT1 cells (=10⁵ cells) in wells of a 96-well plate (Costar Cat. #3603) at 37° C. for 5 hours, then adding 20 μl of 6X CCF2-AM loading solution (prepared according to Aurora Biosciences' instructions; final [CCF2-AM]=1 μM) to each well. Cells were incubated with loading solution overnight and fluorescence emissions were measured using a microplate-reading fluorometer. The results of this experiment are presented in Table I.

Table I shows blue fluorescence values in target cells incubated with various supernatants prior to loading with CCF2-AM. Target cells incubated with VSV-G-containing particles displayed increased blue fluorescence, indicating the presence of β-lactamase in the cells, while target cells incubated with particles lacking an envelope glycoprotein or generated in the presence of HXB2 gp160 displayed only background levels of blue fluorescence.

TABLE I

| HIVRP generated by transfecting 293T | Blue Fluorescence Units |
|---|---|
| cells with pMM304 + pMM310 + | In Target Cells |
| No envelope glycoprotein | 5044 |
| VSV-G protein | 39236 |
| HXB2 gp160 | 9280 |

Epifluorescence observation confirmed that most of the cells incubated with VSV-G-containing particles appeared blue, while cells incubated with other particles appeared mostly green. The results indicate that transfer of β-lactamase to target cells required that virions be generated in cells coexpressing both an envelope glycoprotein (e.g., VSV-G) and β-lactamase-Vpr. The requirement for an envelope glycoprotein suggests that transfer of β-lactamase to target cells is a result of VSV-G-mediated particle entry.

Replication Deficient VSV-G Reporter Particle

Entry competent VSV-G reporter particles made replication-incompetent were generated by cotransfection using the calcium phosphate procedure. In brief, a confluent flask of 293T cells was treated with trypsin/EDTA solution to remove cells, and 1/7 of the cells were plated into each of 4 Costar 10 cm tissue culture dishes. The following day, cells were transfected with DNA mixes as follows:

Flask 1: 15 μg pMM304+5 μg pMM310+5 μg pCMV-VSVG

Flask 2: 15 μg pMM304+5 μg pMM310+5 μg HXB2 gp160 plasmid

Flask 3: 15 μg pMM312+5 μg pMM310+5 μg pCMV-VSVG

Flask 4: 15 μg pMM312+5 μg pMM310+5 μg HXB2 gp160 plasmid

Each DNA mix (20 μg) was diluted in water to 440 μl, then 60 μl of 2.5 M CaCl2 solution were added (from kit).

To form CaPO₄ precipitates, these solutions were added dropwise to 0.5 ml of HEPES-buffered saline solution (from kit) with vigorous agitation and incubated 30 minutes. Each DNA precipitate was added dropwise to one dish of 293T cells. After overnight incubation, cells were washed with phosphate-buffered saline and then incubated 2 additional days with fresh medium.

Culture supernatants were harvested and tested essentially as described in the previous section. Table H shows that both supernatants from cells transfected with either pMM304 or pMM312 are capable of transferring β-lactamase to target cells only when the transfected cells also expressed the VSV-G protein. Transfection of an HXB2 gp160 expression plasmid did not yield supernatants capable of transferring a significant level of β-lactamase to target cells.

TABLE II

| | Blue/Green Fluorescence |
|---|---|
| HIVRP added to target cells: | Ratio in Target Cells |
| Medium only | 0.024 |
| pMM304 + pMM310 + VSV-G | 1.41 |
| pMM304 + pMM310 + HXB2 gp160 | 0.109 |
| pMM312 + pMM310 + VSV-G | 3.099 |
| pMM312 + pMM310 + HXB2 | 0.088 |

Blocking Entry of VSV-G Particles

Virus entry directed by the VSV-G protein is sensitive to lysosomotropic agents such as NH₄Cl. To confirm that β-lactamase was being transferred to target cells by means of legitimate VSV-G-driven virus entry, cells were incubated with VSV-G-enveloped particles in the continual presence or absence of 10 mM NH₄Cl. By fluorescence microscopy, it could be observed that cultures incubated with particles in the presence of NH₄Cl contained significantly fewer blue cells than did cultures incubated in the absence of NH₄Cl. Estimations of percentages of blue cells based on fluorescence micrographs are presented in Table III. The results in Table III confirm that transfer of β-lactamase requires a functional virus entry pathway.

TABLE III

| pMM312 + pMM310 + | pMM312 + pMM310 + |
|---|---|
| pCMV − VSVG | pCMV − VSVG + |
| w/o NH₄Cl | 10 mM NH₄Cl |
| ˜80-90% blue cells | ˜10% blue cells |

Example 3: HIV Reporter Particles Containing HIV Envelope Glycoprotein

Viral reporter particles were generating using the β-lactamase-vpr expression plasmid pMM310 and the wild-type HIV proviral DNA designated pNL4-3. Transfections of 293T cells by the calcium phosphate method were done essentially as described in Example 2, with the following modifications: i) 1.5×10⁶ 293T cells were plated in each 10 cm dish; ii) for CaPO4 precipitate formation, a total of 25 μg of DNA (with various ratios of pMM310 DNA to pNLA-3 DNA) were transfected using 62 μl of 2 M CaCl₂ and 0.5 ml of HEPES-buffered saline in a total of 1 ml.

Supernatants were harvested and tested as described in Example 2 for the ability to transfer β-lactamase to SupT1 target cells. After a 5 hour incubation of target cells and supernatants at 37° C., cells were loaded with CCF2-AM and incubated overnight at room temperature. By epifluorescence microscopy, it was observed that pNIA-3/pMM310 supernatants were able to transfer β-lactamase to ˜5-10% of cells (i.e., blue fluorescent cells). Different ratios of pNLA-3 to pMM310 all produced similar results, and, in contrast with the VSV-G-pseudotyped particles, the inclusion of 10 mM NH4Cl did not block transfer of β-lactamase.

Estimations of percentages of blue cells based on fluorescence micrographs are presented in Table IV. The results shown in Table IV illustrate the ability of HIV reporter particles to enter cells by the normal pathway of HIV target cell entry via gp120/gp41-driven membrane fusion.

TABLE IV

| pNL4-3 (5 μg) + | pNL4-3 (5 μg) + | pNL4-3 (5 μg) + | pNL4-3 (5 μg) + |
|---|---|---|---|
| pMM310 (5 μg) | pMM310 (5 μg) + | pMM310 (20 μg) | pMM310 (20 μg) + |
| w/o NH₄Cl | 10 mM NH₄Cl | w/o NH₄Cl | 10 mM NH₄Cl |
| ˜5-10% blue cells | ˜10% blue cells | ˜5-10% blue cells | ˜10% blue cells |

The ability of HIV reporter particles to enter a cell by means of gp120/gp41-driven fusion, and use of HIV reporter particles in an entry inhibition assay, was confirmed using known glycoprotein inhibitors. NLA-3/pMM310generated HIV reporter particles were incubated with target cells in the presence or absence of specific inhibitors. Both DP-178 (a gp41 inhibitor) and IgGlb12 (a gp120 inhibitor) blocked the transfer of β-lactamase to target cells by NIA-3-derived HIV reporter particles, but neither agent blocked transfer of β-lactamase to target cells by VSV-G-bearing HIV reporter particles.

Formation of entry competent HIV reporter particles was inhibited using a protease inhibitor. pNL4-3-derived HIV reporter particles were generated by transfecting each 10 cm dish of 293T cells with 10 μg each of pNL4-3 and pMM310 using the calcium phosphate method described in Example 2. In one transfection, the HIV protease inhibitor indinavir was included continuously in the culture medium at a concentration of 1 μM. Supernatants were harvested and tested for entry-competent HIV reporter particle as described in Example 2.

As observed by epifluorescence microscopy, supernatants of HIV reporter particles generated in the absence of inhibitor transferred 0-lactamase to ˜10-20% of target cells. However, those HIV reporter particles generated in the presence of indinavir were unable to transfer β-lactamase to target cells efficiently (˜1%).

Estimations of percentages of blue cells based on fluorescence micrographs are presented in Table V. The results in Table V indicate that only mature HIV virions are competent to enter target cells and further indicates that the transfer of β-lactamase to target cells is mediated by the authentic viral entry pathway.

TABLE V

| | PNL4-3 + pMM310 | PNL4-3 + pMM310 |
|---|---|---|
| pNL4-3 + pMM310 | Made in presence of | Made in presence of |
| made w/o inhibitor | 1 μM indinavir | 1 μM L-697661 |
| ˜10-20% blue cells | ˜1% blue cells | ˜10-20% blue cells |

Example 4: Generation of HIV Reporter Particles using Different Proviral Clones

This example illustrates the construction of HIV reporter particles using different HIV proviral clones. HIV reporter particles were prepared from YU2 and R8 strains.

Reporter particles produced from the YU2 strain were generated by transfecting 293T cells (10 cm dish) with 10 μg of pYU2 or pNLA-3 along with 10 μg of pMM310 using the calcium phosphate method described in Example 2. Culture supernatants from the transfected cells were harvested and tested for entry-competent HIV reporter particle as described in Example 2 except that target cells were SupT1 cells stably expressing the CCR5 protein, which is required for entry by YU2 virions. Observation of CCF2-loaded cells by epifluorescence microscopy revealed that supernatants containing NLA-3-derived HIV reporter particle transferred β-lactamase to ˜10-20% of target cells. Supernatants containing YU2-derived HIV reporter particle also transferred β-lactamase to target cells, but a smaller fraction of the target cells appeared blue.

Reporter particles produced from the R8 strain were generated by transfecting 293T cells (10 cm dish) with 10 μg of R8 along with 10 μg of pMM310 using the calcium phosphate method described in Example 2. Culture supernatants from the transfected cells were harvested and tested for entry-competent HIV reporter particles as described above using CCR5-expressing SupT1 cells as targets.

Observation of CCF2-loaded cells by epifluorescence microscopy revealed that supernatants containing R8-derived HIV reporter particle transferred lactamase to ˜70-80% of target cells. Estimations of percentages of blue cells based on fluorescence micrographs are presented in Table VI.

TABLE VI

| pNL4-3 + pMM310 | R8 + pMM310 |
|---|---|
| ˜10% blue cells | ˜70-80% blue cells |

The HIV reporter particle derived from the R8 provirus consistently transferred β-lactamase to target cells more efficiently than did HIV reporter derived from other provirus DNAs that were tested. In an embodiment of the present invention, the reporter particle is based on R8.

Example 5: Different Vpr and β-lactamase Constructs

Several different configurations of fusions between β-lactamase and Vpr were constructed and tested for the ability to generate HIV reporter particles when coexpressed with HIV proteins. Variations tested included changes in the orientation of the fusion (i.e., Vpr-β-lactamase or β-lactamase-Vpr), the presence or absence of a synthetic HIV protease cleavage site between the β-lactamase and Vpr moieties, and the choice of promoter. Four representative constructs tested were:

pMM307: vpr-BlaM w/SV40 promoter
pMM308: BlaM-vpr w/SV40 promoter
pMM310: BlaM-vpr w/CMV promoter
pMM311: BlaM-PR-vpr w/CMV promoter

The four constructs were tested at the same time by cotransfecting one 10 cm dish of 293T cells with 10 μg of each test plasmid along with 10 μg of the proviral DNA NL4-3/pRL500 using the calcium phosphate procedure described in Example 2. Culture supernatants were generated and tested for entry competence using SupT1/CCR5 cells as targets.

Observation of CCF2-loaded cells by epifluorescence microscopy revealed that supernatants containing HIV reporter particle made by cotransfection of pRL500 and pMM310 transferred β-lactamase to ˜25% of target cells. By contrast, supernatants made from cells cotransfected with pRL500 and any of the other Vpr-β-lactamase fusion constructs transferred β-lactamase to only a small number of cells. Estimations of percentages of blue cells based on fluorescence micrographs are presented in Table VII. Taken together, the data indicate that efficient HIV reporter particle production is facilitated by expression from a strong promoter (e.g., CMV) of a β-lactamase-Vpr construct lacking a protease site.

TABLE VII

| PRL500 + | PRL500 + | pRL500 + | pRL500 + |
|---|---|---|---|
| PMM307 | pMM308 | pMM310 | pMM311 |
| 0% blue cells | 0% blue cells | ˜25% blue cells | A few blue cells |

Example 6: Entry Competent Reporter Particles Need Not Be Competent To Complete Later Steps In The Virus Life Cycle

Entry competent reporter particles need not be competent to complete post-entry steps in the HIV life cycle (e.g., reverse transcription, integration). Thus, useful viral reporter particles can be produced lacking, or with altered, genes involved in post-entry activities.

HIV reporter particles were generated by cotransfecting 293T cells with 10 μg each of the NLA-3 proviral plasmid and plasmid pMM310 as described in Example 2. Culture supernatants were then tested for the ability to transfer β-lactamase to SupT1/CCR5 target cells as described in Example 4, but either in the absence or presence of 1 μM of reverse transcriptase inhibitor L-697661. At this concentration, L-697661 completely blocks synthesis of full-length HIV cDNA in cells.

As observed by epifluorescence microscopy, inclusion of 1 μM L-697661 in the virus entry assay had no effect on the ability of HIV reporter particle to transfer β-lactamase to target cells. Estimates of the percentage of blue cells in various conditions are presented in Table VIII.

TABLE VIII

| NL4-3 | NL4-3 + pMM310 | NL4-3 + pMM310 |
|---|---|---|
| w/o inhibitor | w/o inhibitor | + 1 μM L-697661 |
| 0% blue cells | 10-20% blue cells | 10-20% blue cells |

The HIV proviral plasmid (pRL500) was derived from the pNLA-3 HIV molecular clone and encodes a mutant HIV unable to complete the integration step. Upon transfecfion, this proviral plasmid yields virus particles incompetent for integration and unable to establish a spreading infection in tissue culture. (LaFemina et al., J. Virol. 66:7414-7419, 1992.)

HIV reporter particles were made by cotransfecting 293T cells with pMM310 and either pRL500 or pNL4-3 by the calcium phosphate method as described in Example 2. Culture supernatant were harvested and tested for entry competence using the SupT1/CCR5 target cells. As observed by epifluorescence microscopy, both the wild-type pNIA-3 and the integration-defective mutant pRL500 yielded HIV reporter particles to transfer β-lactamase to target cells with similar efficiency (˜10-20% blue cells in each case).

Example 7: Using Reporter Particles in an Entry Inhibition Assay

The present invention can be used to identify and determine the potency of HIV entry inhibitors. In this example, two different HIV reporter particles were tested, one generated from the R8 HIV provirus and one generated from the R8.BaL provirus.

HIV reporter particles were generated by cotransfecting 293T cells with 10 μg of provirus plasmid and 10 μg of pMM310 using the calcium phosphate method described in Example 2. Supernatants were tested using SupT1/CCR5 target cells as described in Example 4, except that various concentrations of inhibitor were present during the incubation of target cells with HIV reporter particles.

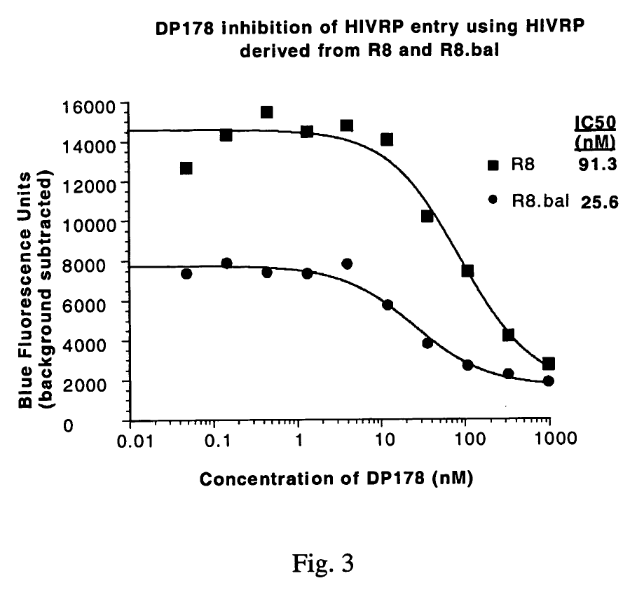

Increasing concentrations of the peptide DP-178 in cultures of HIV reporter particles and target cells resulted in a dose-dependent decrease of the magnitude of blue fluorescence as measured in a fluorometer (FIG. 3). Concurrent observation by epifluorescence microscopy revealed that the presence of increasing concentrations of inhibitor resulted in a dose-dependent decrease in the number of blue cells. These results are consistent with DP-178 inhibition of gp120/gp41-driven virion entry. Analysis of the data by non-linear curve fitting to a 3 parameter logistic equation indicated that the IC₅₀ (concentration of inhibitor needed to inhibit 50% of the signal) for the R8 and R8.BaL HIV reporter particle preparations were 91nM and 26nM, respectively.

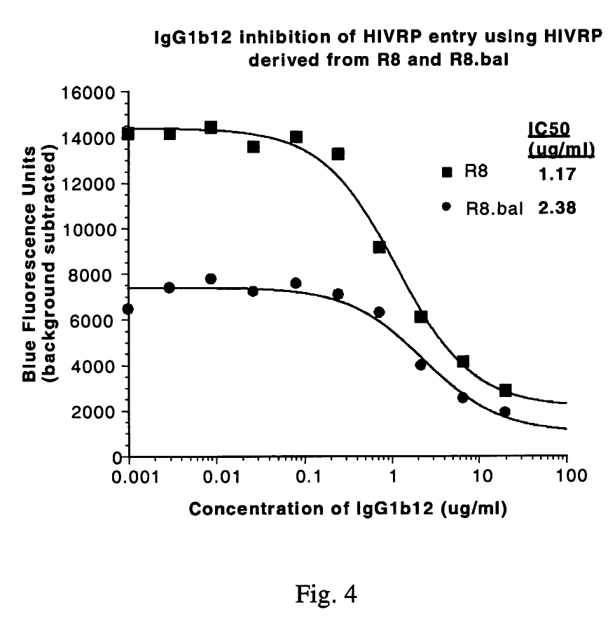

Increasing concentrations of the human antibody IgGlbl2 in cultures of HIV reporter particle and target cells resulted in a dose-dependent decrease of the magnitude of blue fluorescence as measured in a fluorometer (FIG. 4). Concurrent observation by epifluorescence microscopy revealed that the presence of increasing concentrations of inhibitor resulted in a dose-dependent decrease in the number of blue cells. These results are consistent with IgGlb12 inhibition of gpl2O/gp41-driven virion entry. Analysis of the data by non-linear curve fitting indicated that the IC₅₀ (concentration of inhibitor needed to inhibit 50% of the signal) for the R8 and R8.BaL HIV reporter particle preparations were 1.2 μg/ml and 2.4 μg/ml, respectively.

Example 8: Pseudotyping with AMLV Glycoprotein

To investigate whether envelope virus glycoproteins from other viruses could be incorporated functionally into IRV reporter particles, 293T cells were cotransfected with the following DNAs:

1. 10 μg R9 PR Δenv+10 μg of pMM310

2. 10 μg R9 PR Δenv+10 μg of pMM310+5 μg pCMV-VSVG

3. 10 μg R9 PR Δenv+10 μg of pMM310+5 μg pSV-AMLV

HIV reporter particles were harvested as described in Examples 2.

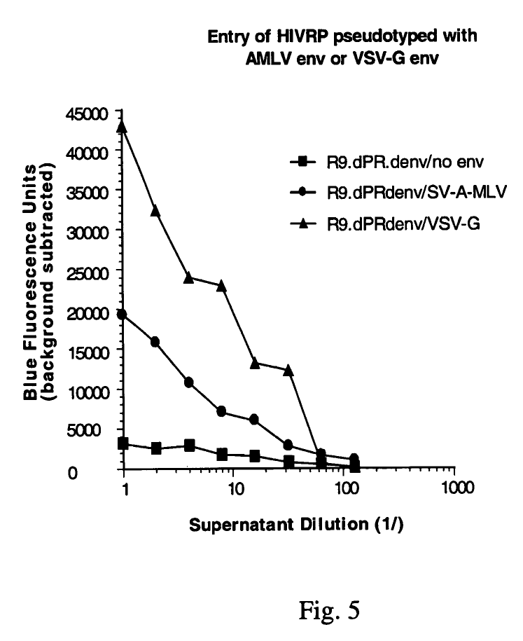

Serial 2-fold dilutions of the HIV reporter particles containing supernatants were tested for entry by incubating with SupT1/CCR5 cells for 5 hours at 37° C., then cells were loaded with CCF2-AM as described in Example 4. As shown in FIG. 5, HIV reporter particles lacking an envelope glycoprotein failed to transfer β-lactamase to target cells.

HIV reporter particles bearing either the VSV-G or the AMLV envelope glycoprotein transferred βlactamase to target cells in an HIV reporter particle dose-dependent manner. By both fluorometric and microscopic analysis, the VSV-G protein supported entry into a greater number of cells than did the AMLV protein. Nevertheless, the observation that the AMLV directed entry of HIV reporter particles into some target cells provides a demonstration and second example indicating that envelope glycoproteins from different viruses can function when incorporated into HIV reporter particles.

Example 9: Incorporation of Envelope Glycoproteins from Primary (Clinical) HIV Isolates into Reporter Particles

HIV reporter particles incorporating glycoproteins using the gp160 genes from primary HIV isolates were produced. The HIV R8 genome was used to construct the reporter particles.

The R8 genome contains several unique restriction sites present toward the 3 ′ end of the genome (i.e., BamHI, CelII, and XhoI) which are often present in primary ERV-1 genomes. To allow insertion of gp160 genes from primary HIV-1 isolates into the R8 genome, the R8 provirus DNA clone was modified by installation of a unique recognition site for the endonuclease NotI just 5 ′ of the translation start site of gp160 (plasmid pMM326).

Primary gp160 genes were amplified by polymerase chain reaction (PCR) using the Expand High-fidelity PCR system according to the manufacturer's instructions (Roche). Oligonucleotides for the PCR amplification were the downstream primer pMM440 and an upstream primer MM439, which includes a NotI site. DNA templates consisted of genomic DNA isolated from PBMCs infected with primary HIV isolates 1021, 1022, and 1036. Amplification conditions were essentially as described in Gao et al., J. Virol. 70:1651-1667, 1996. The amplification products were digested with NotI and either CelII or XhoI and ligated into pMM326 digested with the same enzymes. The resulting plasmids are designated R8.1021, R8.1022, and R8.1036.

HIV reporter particles were generated by transfecting 293T cells with pMM310 and each of the HIV provirus plasmids R8, R8.BaL, R8.1021, R8.1022, and R8.1036 using the calcium phosphate method described in Example 2. Supernatants were harvested as described in Example 2 and tested for entry by incubating 90 μl of supernatant with SupT1/CCR5 target cells (10⁵ in 10 μl) in the presence or absence of the specific inhibitor DP-178. Target cells were incubated with supernatants at 37° C. for 5 hours, then loaded with 1 μM CCF2-AM overnight at room temperature.

By epifluorescence microscopy, it was observed that plasmids R8, R8.BaL, R8.1021, and R8.1036 efficiently transferred β-lactamase to SupT1/CCR5 cells. Results of fluorometric analysis are shown in Table IX.

TABLE IX

| | |
|---|---|
| HIVRP generated by | Blue/Green Fluorescence in Target |
| transfection with | cells incubated with HIVRP with: |

| PMM310 + | No inhibitor | 1 μM DP178 |
|---|---|---|
| R8 | 0.84 | 0.13 |
| R8.1021 | 0.97 | 0.076 |
| R8.1022 | 0.24 | 0.10 |
| R8.1036 | 1.30 | 0.86 |
| R8.bal | 1.34 | 0.097 |

Inclusion of 1 μM DP-178 peptide efficiently blocked entry by all HIV reporter particles except R8.1036; entry of this isolate was blocked efficiently by other inhibitors (data not shown). Collectively, these results show that the present invention allows facile analysis of the entry competence function of gp160s encoded by primary HIV-1 isolates.

Example 10: Use of Primary Human Cells as Target Cells

The results described in this section demonstrate that HIV reporter particles can be used in conjunction with uncloned primary human cells to evaluate HIV entry. HIV reporter particles transferred β-lactamase to human monocyte-derived macrophages and primary peripheral blood mononuclear cells.

PBMCs were isolated from donated blood by standard techniques. Monocytes were obtained from the PBMCs by plastic adherence using standard techniques and were cultured in monocyte/macrophage medium in Teflon jars to differentiate them into macrophages. Macrophages were resuspended at 10⁷ cells/ml in phenol red-free DMEM with 10% FBS. Cells (10 μl=10⁵ cells) were incubated 20 with 90 μl of either R8 or R8.BaL HIV reporter particle supernatants for 4 hours at 37° C. and then loaded with 1 μM CCF2-AM overnight at room temperature.

By light microscopy, cultures contained both large, flat adherent cells and small, round non-adherent cells. Observation by epifluorescence microscopy revealed that both R8- and R8.BaL-derived HIV reporter particle were able to transfer β-lactamase to cells in the culture, indicating that primary cells can be entered by HIV reporter particles.

It was further evident that R8-derived HIV reporter particles transferred β-lactamase preferentially to the small round cells, while R8.BaL-derived HIV reporter particle transferred β-lactamase preferentially to the large adherent cells. These observations are consistent with the previously published observation that the R8 envelope tends to direct entry of viruses into T cells (T tropic) while the BaL envelope tends to direct entry of viruses into macrophages (M tropic).

In another experiment, PBMCs were isolated from the blood of 4 different donors. Blood was collected by venipuncture into EDTA-containing Vacutainer tubes, and PBMCs were prepared by standard techniques. PBMCs were resuspended at 10⁷ cells/ml in phenol red-free DMEM with 10% FBS. Cells (10 μl=10⁵ cells) were incubated with 90 μl of either R8 or R8.BaL HIV reporter particle supernatants for 4 hours at 37° C. in the absence or presence of 1μM DP-178. After this incubation, cells were loaded with 1 μM CCF4-AM overnight at room temperature.

Observation of cells by epifluorescence microscopy indicated that both R8-derived and R8.BaL-derived HIV reporter particles transferred β-lactamase to PBMCs from all four donors. In the absence of inhibitor, ˜20-25% of cells from each donor appeared blue after incubation with either type of HIV reporter particle. The ability of DP-178 to inhibit β-lactamase transfer to PBMCs indicates that transfer was mediated by gp120/gp41.

Example 11: Additional Transfection Techniques

HIV reporter particles can be produced by transfecting cells by methods other than the calcium phosphate precipitation. To optimize transfection conditions to produce HIV reporter particles, various commercially available transfection kits were tested. In each case, 293T cells (1.5×10⁶ cells seeded the previous day in a 10 cm dish) were transfected according to manufacturer's recommendations using 5 μg of R8 DNA and 5 μg of either pMM310 or an irrelevant DNA.

Transfections were done overnight with calcium phosphate, Fugene6 (60 μl), Effectene (16 μl of enhancer), or TransIT (50 μl of transfection reagent). The following day the culture medium was removed, cells were washed once with 10 ml of PBS, and cells were refed with 8 ml of phenol red-free DMEM/10% FBS and incubated for 48 hours. Supernatants were harvested as described in Example 2 then tested in entry assays by incubating serial 2-fold dilutions of supernatants (90 μl/well) with SupT1/CCR5 cells (10 μl=10⁵ cells/well) in a 96-well plate at 37° C. as described in Example 4.

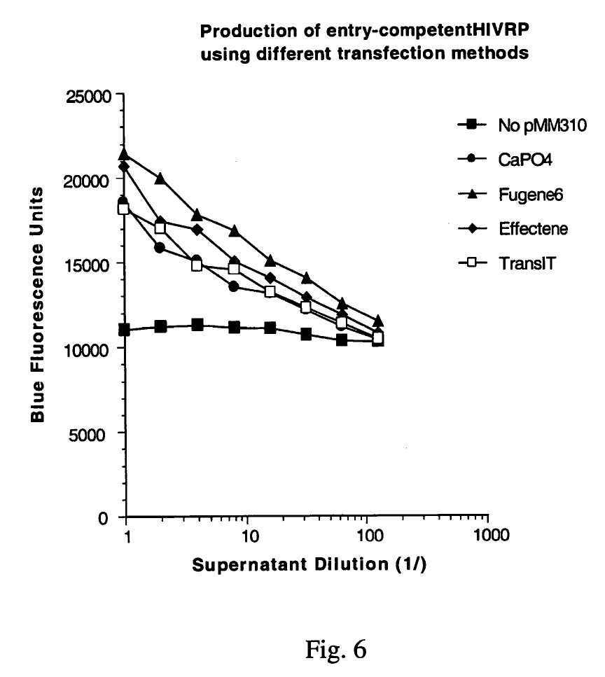

After the incubation, cells were loaded with CCF2-AM overnight at room temperature, then fluorescence was measured using a BMG PolarStar fluorometer. Results shown in FIG. 6 indicate that all transfection methods produced entry-competent HIV reporter particles.

Other embodiments are within the following claims. While several embodiments have been shown and described, various modifications may be made without departing from the spirit and scope of the present invention.

What is claied is:

1. A chimeric protein comprising a first region that is a β-lactamase region and a second region that is either a Vpr region or a Vpx region, wherein said second region is on the carboxy side of said first region, said chimeric protein can be packaged in an entry competent lentiviral particle, and said chimeric protein has β-lactamase activity.

2. The chimeric protein of claim 1, wherein said entry competent lentiviral particle is a human immunodeficiency virus.

3. The chimeric protein of claim 2, wherein said chimeric protein does not contain any human immunodeficiency virus protease recognition sites between said first region and said second region.

4. The chimeric protein of claim 3, wherein said first region is said Vpr region.

5. The chimeric protein of claim 3, wherein said first region is said Vpx region.

6. The chimeric protein of claim 3, wherein said chimeric protein consists of the amino acid sequence of SEQ. ID. NO. 2.

7. An expression vector comprising nucleic acid expressing the chimeric protein of any one of claims 1-6.

8. An entry competent viral reporter particle comprising the chimeric protein of any one of claims 2-6,
one or more viral envelope glycoproteins,
a lipid bilayer,
a human immunodeficiency virus matrix capsid,
a human immunodeficiency virus capsid,
a human immunodeficiency virus nucleocapsid, and
a human immunodeficiency virus C-terminal p6 domain.

9. The viral particle of claim 8, wherein said one or more envelope glycoproteins is vesicular stomatitis virus G glycoprotein.

10. The viral particle of claim 8, wherein said one or more envelope glycoproteins are HIV gp120 and HIV gp41.

11. The viral particle of claim 10, wherein said particle is replication incompetent.

12. The viral reporter particle of claim 10, wherein said HIV gp120 is CCR5 tropic.

13. The viral reporter particle of claim 12, wherein said HIV gp120 is from human immunodeficiency virus Bal, JRFL, SF162, or YU2.

14. The viral reporter particle of claim 10, wherein said HIV gp120 is CXCR4 tropic.

15. The viral reporter particle of claim 14, wherein said HIV gp120 is from human immunodeficiency virus NMA-3, R8 or MN.

16. The viral reporter particle of claim 10, wherein said one or more envelope glycoproteins are produced from gp160 obtained from a primary human immunodeficiency virus isolate.

17. The viral reporter particle of claim 8, wherein said one or more envelope glycoproteins is murine leukemia virus envelope glycoprotein.

18. The viral reporter particle of claim 8, wherein said one or more envelope glycoproteins are HCV E1 and E2.

19. The viral reporter particle of claim 8, wherein said human immunodeficiency virus matrix capsid, said human immunodeficiency virus capsid, said human immunodeficiency virus nucleocapsid, and said human immunodeficiency virus C-terminal p6 domain are all from HIV R8.

20. An entry competent viral reporter particle made by a process comprising the steps of:
a) cotransfecting a cell with one or more nucleic acids expressing the chimeric protein of any one of claims 1-6 and components needed to produce an entry competent viral reporter particle containing one or more envelope glycoproteins; wherein said chimeric protein is packaged by said viral reporter particle and has β-lactamase activity; and
b) growing said cell cotransfected in step (a) under viral production conditions to produce said viral particle.

21. The viral reporter particle of claim 20, wherein said one or more nucleic acids are present on one or more expression vectors.

22. The viral reporter particle of claim 21, wherein said components are from HIV R8.

23. The viral reporter particle of claim 21, wherein said process further comprises the step of purifying said viral particle.

24. The viral reporter particle of claim 21, wherein said one or more envelope glycoproteins is vesicular stomatitis virus G glycoprotein.

25. The viral reporter particle of claim 21, wherein said one or more envelope glycoproteins are HIV gp120 and HIV gp41.

26. The viral reporter particle of claim 25, wherein said HIV gp120 is CCR5 tropic.

27. The viral reporter particle of claim 26, wherein said HIV gp120 is from human immunodeficiency virus Bal, JRFL, SF162, or YU2.

28. The viral reporter particle of claim 25, wherein said HIV gp120 is CXCR4 tropic.

29. The viral reporter particle of claim 28, wherein said HIV gp120 is from human immunodeficiency virus NL4-3, R8 or MN.

30. The viral reporter particle of claim 21, wherein said one or more envelope glycoproteins are produced from gp160 obtained from a primary human immunodeficiency virus isolate.

31. The viral reporter particle of claim 21, wherein said one or more envelope glycoproteins is murine leukemia virus envelope glycoprotein.

32. The viral reporter particle of claim 21, wherein said one or more envelope glycoproteins are HCV E1 and E2.

33. A method of measuring the ability of a compound to inhibit viral entry into a cell comprising the steps of:
a) combining together (i) an entry competent viral reporter particle comprising the chimeric protein of any one of claims 1-6, (ii) a target cell, and (iii) said compound, under conditions allowing entry of said viral particle into said target cell in the absence of said compound; and
b) measuring β-lactamase activity in a host cell as a measure of the ability of said compound to inhibit viral entry.

34. The method of claim 33, wherein said target cell is a primary human cell.

35. The method of claim 33, wherein said viral reporter particle is an R8 provirus.

36. A method of measuring the ability of a compound to inhibit mature virus production comprising the steps of:
a) growing a recombinant cell able to produce a viral reporter particle comprising the chimeric protein of any one of claims 1-6 under viral production conditions in the presence of said compound, and
b) measuring the production of entry competent viruses in step (a) that can provide β-lactamase activity to a host cell as an indication of the ability of said compound to inhibit mature virus production.

37. The method of claim 36, where said recombinant cell comprises one or more expression vectors that together express said chimeric protein and components needed to produce an entry competent viral reporter particle containing one or more envelope glycoproteins.

38. The method of claim 37, wherein said viral reporter particle is an R8 provirus.

39. The viral particle of claim 37, wherein said one or more envelope glycoproteins is vesicular stomatitis virus G glycoprotein.

40. The viral particle of claim 37, wherein said one or more envelope glycoproteins are HIV gp120 and HIV gp41.

41. The viral particle of claim 37, wherein said one or more envelope glycoproteins are HIV E1 and E2.

42. The viral particle of claim 37, wherein said one or more viral envelope glycoproteins is murine leukemia virus envelope glycoprotein.
